(19) United States
(12) Patent Application Publication
(10) Pub. No.: US 2005/0107303 A1
(43) Pub. Date: May 19, 2005

(54) Health feed

(76) Inventors: Richard Fulton Butterwick, Waltham-on-the-Wolds (GB); Vivien Elizabeth Rolfe, Waltham-on-the-Woods (GB); Charlene Elizabeth Vallance, Waltham-on-the-Woods (GB)

Correspondence Address: FULBRIGHT & JAWORSKI, LLP, 1301 MCKINNEY, SUITE 5100, HOUSTON, TX, 77010-3095, US

(21) Appl. No.: 10/480,373

(22) PCT Filed: Jun. 13, 2002
(86) PCT No.: PCT/GB02/02752

(30) Foreign Application Priority Data
Jun. 13, 2001 (GB) 0114419.5

(51) Int. Cl. A61K 31/198; A61K 38/06; A61K 38/05; A61K 38/04

(52) U.S. Cl. 514/17; 514/563; 514/18; 514/19

(57) ABSTRACT

The present invention relates to a method of promoting immunoglobulin A secretion in the mucosal membranes of non-human animals. The method comprises administering a foodstuff comprising glutamine to the non-human animal.

### CROSS-REFERENCE TO RELATED APPLICATIONS

This application is the National Stage Application of International Application No. PCT/GB02/0752 filed Jun. 13, 2002, which claims priority to Great Britain Application No. 0114419.5 filed Jun. 13, 2001, which are incorporated herein in their entirety.

### TECHNICAL FIELD

The present invention relates to a method of promoting immunoglobulin A secretion in the mucosal membranes of non-human animals. The method comprises administering a foodstuff comprising glutamine to the non-human animal.

### BACKGROUND OF THE INVENTION

Glutamine is a neutral amino acid, which is readily transported across plasma membranes. As an important intermediate in a number of metabolic pathways, cellular utilization of glutamine can far exceed that of other amino acids especially within intestinal and immune cells. Glutamine is important in the transport of amino nitrogen and ammonia, as a substrate in gluconeogenesis and ammoniagenesis, as a fuel source for rapidly dividing cells and may also be involved in the regulation of protein synthesis.

The high rate of glutamine utilization by the intestine (identified to occur in man) may be due in part to the large lymphocyte and macrophage populations in intestinal walls and Peyer's patches. These cells exhibit high glutaminase activity and utilize glutamine as their preferential fuel source, even in the quiescent state. However, as with many other cells that require glutamine, both enterocytes and lymphocytes lack the synthetic apparatus to produce glutamine relying solely on circulatory or dietary sources. It is suggested that a fall in the plasma concentration could compromise lymphocyte function accounting for the increase in susceptibility to viral infection.

Gut-associated lymphoid tissue in the gastrointestinal tract appears to provide immunologic protection for the gastrointestinal tract and for extra intestinal mucosal sites such as the nasopharynx, mammary glands, salivary glands and lungs. Lymphocytes, which provide or control the production of immunoglobulin A are released by the gut-associated lymphoid tissue and distributed to the gastrointestinal tract and extra intestinal sites via the mesenteric lymphatic channels and thoracic duct. Feeding of an individual by total parenteral nutrition results in the atrophy of gut-associated lymphoid tissue and a decrease in immunoglobulin A levels. Addition of glutamine to a total parenteral nutrition solution was shown by Li et al to normalize gut-associated lymphoid tissue population.

Work by Gismondo et al (Gismondo, M. R., Drago, L., Fassina, M. C., Vaghi, I., Abbiati, R. and Grossi, E. (1998). Immunostimulating effect of oral glutamine. Dig. Dis. Sci, 43, 1752-1754) has indicated that the oral administration of glutamine to congenitally immunosuppressed mice provides a positive effect on serum levels of interleukin 2 and the intestinal population of T cells.

In addition, studies in man by Fujita and Sakura (Fujita, T. and Sakurai, K. (1995) Efficacy of glutamine-enriched enteral nutrition in an experimental model of mucosal ulcerative colitis. Br. J. Surg, 82, 749-751) have indicated that the administration of glutamine is therapeutically beneficial to patients with inflammatory bowel disease.

It is a desire in the area of pet food products and companion animal health as well as farm animal health to provide diets suitable to support the health of non-human animals. In particular it is desire to provide diets suitable to promote or maintain the health of already healthy non-human animals.

### BRIEF SUMMARY OF THE INVENTION

The first aspect of this invention relates to a method of promoting immunoglobulin secretions in the mucosal membranes of a non-human animal comprising administering a foodstuff comprising glutamine to a non-human animal.

Mucosal membranes are the moist membranes lining many tubular structures and cavities. These membranes provide a protective layer between the external environment and the internal organs of an animal. Mucosal cells/tissues include mucosal coverings of the gut, the mouth, the nasal passage, the esophagus, the stomach, the lung, the small intestine, the large intestine, epithelial tissue, urogenital tract, the eyes, and mammary glands.

The method of the first aspect seeks to improve and maintain the health of a non-human animal. In particular, the animals of the first aspect of the invention are pet or companion animals such as cats or dogs or farm animals such as swine (porcine), sheep (ovine) or cattle (bovine) or poultry. The animals may be at any life-stage, such as young, adult or senior. Accordingly, kittens, puppies, piglets, lambs, calves and chicks are covered by the present invention. The maintenance and improvement of the health of a pet or companion animal and of other animals, such as farm animals is a constantly ongoing aim in the art.

It is possible to monitor the improved health of an animal as achieved by the invention in a number of ways. Two of these are feces quality and gastrointestinal (GI) tract health. By improving the health of the animal, the invention seeks to promote and maintain good quality feces in animals (such as pet animals). Good feces quality is of two-fold importance. Firstly, it is a good indicator of a healthy animal (such as a pet). It is known that good feces quality usually reflects healthy colonic structure and function. Secondly, it is a much-favored practicality for pet-owners. The invention also aims to improve the GI tract health of animals (such as pet animals). The ability to maintain and improve GI tract health can be beneficial to animal owners (such as pet owners) because it has an impact on their animal's overall health.

Without being bound by scientific theory, it is believed that by increasing the level of secretary immunoglobulin A in the mucosal membranes, glutamine maintains and promotes health of a non-human animal. It is postulated that elevated levels of immunoglobulin A improve the defense mechanisms of the mucosal membranes by eliminating viruses from epithelial cells and by forming a barrier which prevents the adherence of pathogenic bacteria. For example, elevated levels of immunoglobulin A in the lungs and/or the nasopharynx may protect an animal from micro-organisms that cause influenza or pneumonia. Within the gastrointestinal tract, immunoglobulin A is believed to decrease intestinal permeability and to enhance intestinal absorption. Therefore, a preferred feature of the first aspect of the invention relates to a method for increasing levels of secretory immunoglobulin A in the gastrointestinal tract comprising administering a foodstuff comprising glutamine to the non-human animal. A further preferred feature of the first aspect of the invention relates to a method for increasing the levels of secretory immunoglobulin A in the urogenital tract comprising administering a foodstuff comprising glutamine to a non-human animal.

It is a preferred feature of the invention that the foodstuff of the first aspect is administered to a non-human animal, which is healthy. It is postulated that administering a foodstuff comprising glutamine to a healthy non-human animal will allow the healthy status of that animal to be maintained, as the animal will be less susceptible to viral or bacterial infection.

For the purposes of this invention, ‘health’ is defined as an absence of clinical disease. Thus a healthy animal is an animal which does not exhibit the symptoms of a clinical disease, for example, by its immune status or histology. In a preferred aspect of this invention, the term healthy encompasses animals at optimal health. In another preferred aspect of this invention, the term healthy encompasses animals at optimal or sub optimal health, for example animals with one or more subclinical diseases.

An assessment of the health of a particular animal can be carried out by the owner (i.e. by assessing the quality of the feces of the animal and/or monitoring the appetite of the animal) or by an individual qualified to do so (e.g. a veterinary surgeon, dietician) by assessing the histology and/or immune status of the animal.

The method of the first aspect comprises administering a foodstuff comprising glutamine. The foodstuff may comprise one or more components which provide a source of glutamine such as one or more of gliadin, oat bran, soya bean meal, linseed, cereals, forages, sunflower, lupin, beans, lentils, milk powder, caesin, whey or soy. For the purposes of this invention, cereals include barley, oats, wheat, bran and rye and forages include grass, hay, rye grass etc.

Alternatively, the foodstuff may be supplemented by a source of glutamine. The glutamine source may be the free amino acid (preferably L-glutamine), a peptide rich in L-glutamine or an extract containing L-glutamine. Suitable peptides rich in L-glutamine include dipeptides, tripeptides, tetrapeptides, pentapeptides, hexapeptides, longer chain peptides or peptide mixtures. Such peptide mixtures include proteins rich in L-glutamine, hydrolates or fractions thereof (e.g. peptide mixture(s) obtained from one or more of gliadin, oat bran, soya bean meal, linseed, cereals, forages, sunflower, lupin, beans, lentils, milk powder, caesin, whey or soy). Glutamine can be further provided by an extract containing L-glutamine either as a free amino acid or as a peptide containing L-glutamine (e.g. extracts of gliadin, oat bran, soya bean meal, linseed, cereals, forages, sunflower, lupin, beans, lentils, milk powder, caesin, whey or soy). Other L-glutamine derivatives include L-glutamine salts, N-acyl derivatives of L-glutamine including N-alkanoyl L-glutamine compounds such as N-acetyl L-glutamine. The N-acylation of L-glutamine stabilises the peptide, in comparison with free amino acid L-glutamine. Such peptides may be pH and fluid stable. The dipeptide can be but is not limited to L-alanyl-L-glutamine or L-glycyl-L-glutamine. The dipeptide containing L-glutamine should be stable in solution.

Preferably, the glutamine is provided as the free amino acid L-glutamine or a dipeptide containing L-glutamine. Alternatively, the glutamine is provided either as an extract or peptide mixture from or by wheat gluten or a casein hydrosylate such as sodium caseinate.

Glutamine is preferably provided to the foodstuff at a level of from 0.01% to 10% w/w on a dry matter basis. Preferably the glutamine is provided as free glutamine or a source thereof having an equivalent amount of bioavailable glutamine. Preferably, the glutamine is provided at a level of 0.01% to 5% w/w on a dry matter basis, most preferably, approximately 1% w/w on a dry matter basis or above.

The glutamine of the invention would be provided by the foodstuff at levels of approximately 0.01 g to approximately 1 g per kg body weight per day, more preferably, approximately 0.1 g per kg body weight or above per day. The foodstuff is preferably administered daily, more preferably twice daily. Where the foodstuff is a treat or snack, the foodstuff can be administered one or more times a day, preferably five or more times a day. The amount of glutamine in a foodstuff may therefore vary depending on the number of times a day the foodstuff is to be administered.

The foodstuff of the invention may be a dry product (with approximately 3, 4 or 5 to approximately 15% moisture), a semi-moist product (with approximately 15 to approximately 70% moisture) or a wet product (with approximately 70 to approximately 90% moisture).

The foodstuff according to the present invention encompasses any product that an animal, (such as a pet) consumes in its diet. Thus, the invention covers standard food products as well as food snacks, such as pet food snacks (for example, snack bars, treats, biscuits and sweet products). The foodstuffs preferably a cooked product. It may incorporate meat or animal derived material (such as beef, chicken, turkey, lamb, fish, blood plasma, marrow bone etc or one or more thereof). The product alternatively may be meat free (preferably including a meat substitute such as soya, maize gluten or a soya product) in order to provide a protein source. The product may contain additional protein sources such as soya protein concentrate, milk proteins, gluten etc.

The product may also contain a starch source such as one or more grains (e.g. wheat, corn, rice, oats, barley etc), or may be starch free.

The foodstuff of the invention is preferably produced as a dry product containing from approximately 3%, 4% or 5% to approximately 15% moisture. The preferred dry food is more preferably presented as small biscuit-like kibbles.

The foodstuff is preferably packaged. In this way, the consumer is able to identify, from the packaging, the ingredients in the foodstuff and confirm that it is suitable for the particular animal (such as a pet) in question. The packaging may be metal (usually in the form of a tin or flexifoil), plastic (usually in the form of a pouch), paper or card. The amount of moisture in any product may influence the type of packaging, which can be used or is required.

The foodstuff of the first aspect can be provided as a food supplement. The food supplement can be a powder, sauce, topping, biscuit, kibble, pocket or tablet that can be administered with or without an additional foodstuff. Where the food supplement is administered with an additional foodstuff, the food supplement can be administered sequentially simultaneously or separately. The food supplement may mixed with the foodstuff, sprinkled over the foodstuff or served separately. Alternatively, the food supplement can be added to a liquid provided for drinking such as water or milk.

The foodstuff can be made according to any method known in the art. Foodstuffs for pet animals can be any, including such as in Waltham Book of Dog and Cat Nutrition, Ed. ATB Edney, Chapter by A. Rainbird, entitled “A Balanced Diet” in pages 57 to 74 Pergamon Press Oxford.

The glutamine may be mixed with the other components of the foodstuff or can be added to the completed foodstuff. In a preferred feature of the invention, the glutamine is coated or sprayed on to the surface of the foodstuff. Alternatively, one or more components comprising glutamine are admixed, with one or more other components of the foodstuff.

The second aspect of the invention relates to the use of glutamine in the manufacture of a foodstuff for preventing or treating infection in the gastrointestinal tract.

It is proposed that the administration of the foodstuff will result in an increase in the level of secretary immunoglobulin A in the gastrointestinal tract. It is postulated that such elevated levels of immunoglobulin A will prevent viral or bacterial infection by eliminating viruses from epithelial cells and forming a barrier which prevents adherence of pathogenic bacteria. In addition, elevated immunoglobulin A levels are believed to decrease intestinal permeability and enhance intestinal absorption. Thus the second aspect of the invention further relates to the use of glutamine in the manufacture of a foodstuff for preventing or treating bacterial or viral infections such as calicivirus in the gastrointestinal tract.

All preferred features of the first aspect of the invention also relate to the second aspect.

The third aspect of the invention relates to the use of glutamine in the manufacture of a foodstuff for the promotion or maintenance of the urogenital health of a non-human animal. In particular, the third aspect relates to the use of glutamine in the manufacture of a foodstuff for preventing or treating infection in the urogenital tract of a non-human animal. For the purposes of the third aspect, the infections are preferably bacterial or viral infections of the urogenital tract, such as calicivirus.

All preferred features of the first and second aspects of the invention also relate to the third aspect.

### DETAILED DESCRIPTION OF THE INVENTION

The invention will now be illustrated with reference to the following non-limiting examples.

### EXAMPLES

Composition of a Foodstuff Comprising Glutamine.

The foodstuff is a dry product containing less than 10% water. The foodstuff comprises the following ingredients (˜is approximately).

| | |
|---|---|
| Rice | ˜20% |
| Poultry | ˜23% |
| Maize | ˜17% |
| Maize meal | ˜9% |
| Beef Tallow | ˜10% |
| Glutamine | ˜1% |

All ingredients except glutamine are mixed, cooked and formed into a dry kibble. Glutamine (provided as L-glutamine) is then sprayed onto the external surface of the foodstuff.

### Experimental Data

### Example 1

The study was carried out using eight members of the cat colonic health panel, which were known to be in good (intestinal) health. Cats were wormed and vaccinated 6 months prior to the start of the trial.

The cats underwent the following trial regime;

| | |
|---|---|
| 14 days | Control diet |
| 28 days | Glutamine enriched diet |
| 14 days | Control diet |

Both the control diet and the glutamine enriched diet provided 405 kCal/100 g. The control diet was a dry diet containing approximately 4% water. The control diet comprised the following ingredients (˜is approximately):

| | |
|---|---|
| Poultry | ˜37% |
| Beef Tallow | ˜10% |
| Rice | ˜20% |
| Maize meal and gluten | ˜26% |
| Sunflower oil | ˜3% |
| Brewers yeast and vitamins | ˜3% |

The glutamine-supplemented diet comprised 1% dry weight by weight glutamine provided as L-glutamine. The glutamine was sprayed onto the external surface of the dry control product.

Measurement of Immunoglobulin Production

Levels of immunoglobulin A production by mid colon biopsy samples were measured by enzyme linked immunosorbent assay (ELISA). IgA ELISA test was obtained from Bethyl and performed as described in provided protocol sheets. In short, 96 well microplates were taken and coated with 100 μl anti-canine IgA overnight at 4° C. After each step the plates were washed with ELISA wash (50 Mm) Tris, pH 8.0, 0.1M NaCl, 0.05% Tween 20). Unreacted sites were blocked with tris-buffered saline (TBS) containing 1% bovine serum albumin (BSA) for 30 minutes. Standards and samples were diluted as required, 100 μl added to the plate and incubated for 1 hour at room temperature. After washing, horseradish peroxidase-conjugated anti-canine IgA immunoglobulins were diluted as described in the protocol and incubated on the plate for 1 hour at room temperature. 200 μl of 3,3′,5,5′-tetramethyl benzidine (TMB) was added to the plate as an enzyme substrate and the reaction was stopped with 100 μl of 0.5M H₂SO₄ after 30 minutes. Finally, the absorbence was read spectrophotometrically at 450 nm using an ELISA plate reader.

Results

A significant increase in IgA production by the colon was found in the samples taken from below the transwell when cats were fed diets containing 1% glutamine (P=0.048, GLM) compared to the other three diets. Results are summarized below in the below table.

TABLE
IgA production by biopsy samples

| | IgA production (ng/mg protein) |
|---|---|
| Diet | Intestinal Sample |
| Standard | 569.8 ± 206.7 (a) |
| Glutamine | 1003.1 ± 762.9 (b) |

Same letter denotes no significant different (p > 0.05).

1. A method of promoting immunoglobulin A secretion in a mucosal membrane of a non-human animal comprising administering a foodstuff comprising glutamine to the non-human animal.

2. The method of claim 1, wherein the mucosal membrane is the gastrointestinal tract of the non-human animal.

3. The method of claim 1, wherein in the mucosal membrane is the urogenital tract of the non-human animal.

4. A method as claimed in claim 1 for the use of maintaining or improving the health of a non-human animal.

5. The method of claim 4, wherein the non-human animal is healthy.

6. The method of claim 1, wherein the non-human animal is a companion animal selected from the group consisting of a cat and a dog, or wherein the animal is a porcine, ovine, bovine or poultry animal.

7. The method of claim 1, wherein glutamine is one or more of L-glutamine, a peptide comprising glutamine or an extract comprising glutamine.

8. The method of claim 7, wherein the peptide is one or more of a dipeptide, tripeptide, tetrapeptide, pentapeptide, hexapeptide, a longer chain peptide or a peptide mixture.

9. The method of claim 8, wherein the peptide mixture is derived from one or more of gliadin, oat bran, soya bean meal, linseed, cereals, forages, sunflower, lupin, beans, lentils, milk powder, caesin, whey, soy casein hydrosylate or wheat gluten.

10. The method of claim 1, wherein glutamine is provided to the foodstuff at a level of approximately 1% w/w on a dry matter basis or above.

11. The method of claim 1, wherein the foodstuff contains from approximately 3% to approximately 15% moisture.

12. The method as of claim 1, wherein glutamine is provided to the non-human animal at a level of approximately 0.1 g per kilogram body weight or above per day.

13. The method as of claim 1, wherein the foodstuff is provided one or more times per day.

14. A method of producing a composition for preventing or treating infection in the gastrointestinal tract of a non-human animal comprising the step of providing glutamine to a foodstuff.

15. A method of producing a composition for promoting urogenital health in a non-human animal comprising the step of providing glutamine to a foodstuff.

16. The method of claim 15, wherein the composition prevents or treats infection in the urogenital tract of a non-human animal.

17. The method claim 14, wherein the infection is caused by a virus or a bacteria.

18. The method of claim 14, wherein the non-human animal is healthy.

19. The method of claim 14, wherein the non-human animal is a companion animal selected from the group consisting of a cat and a dog or wherein the animal is a porcine, ovine, bovine or poultry animal.

20. The method of claim 19, wherein glutamine is one or more of L-glutamine, a peptide comprising glutamine or an extract comprising glutamine.

21. The method of claim 20, wherein the peptide is one or more of a dipeptide, tripeptide, tetrapeptide, pentapeptide, hexapeptide, a longer chain peptide or a peptide mixture.

22. The method of claim 21, wherein the peptide mixture is derived from one or more of gliadin, oat bran, soya bean meal, linseed, cereals, forages, sunflower, lupin, beans, lentils, milk powder, caesin, whey, soy, casein hydrosylate or wheat gluten.

23. The method of claim 14, wherein the glutamine is provided to the composition at a level of approximately 1% w/w on a dry matter basis or above.

24. The method of claim 14, wherein the composition contains from approximately 3%, 4% or 5% to approximately 15% moisture.

25. The method of claim 14, wherein the glutamine is provided to the non-human animal at a level of approximately 0.1 g per kilogram body weight or above per day.

26. The method of claim 14, wherein the composition is provided one or more times per day.

27. (canceled)
